(19) United States
(12) Patent Application Publication
(10) Pub. No.: US 2025/0213183 A1
(43) Pub. Date: Jul. 3, 2025

(54) SYSTEM AND METHOD OF MONITORING NOCICEPTION AND ANALGESIA DURING ADMINISTRATION OF GENERAL ANESTHESIA

(71) Applicants: The General Hospital Corporation, Boston, MA (US); Massachusetts Institute of Technology, Boston, MA (US)

(72) Inventors: Patrick L. Purdon, Boston, MA (US); Gustavo Adolfo Balanza Villegas, Boston, MA (US); Kishore M. Bharadwaj, Boston, MA (US); Andrew Mullen, Boston, MA (US); Laura Santa Cruz, Boston, MA (US)

(21) Appl. No.: 18/847,424

(22) PCT Filed: Mar. 16, 2023
(86) PCT No.: PCT/US2023/064571

§ 371 Date: Sep. 16, 2024

Related U.S. Application Data
(60) Provisional application No. 63/320,535, filed on Mar. 16, 2022.

(51) Int. Cl. A61B 5/00; A61B 5/0531; A61B 5/372; G16H 40/63

(52) U.S. Cl. CPC A61B 5/4821; A61B 5/0531; A61B 5/372; A61B 5/7246; G16H 40/63

(57) ABSTRACT

Systems and methods are provided for patient monitoring and/or treatment. The system may include one or more sensors configured to measure electroencephalogram (EEG) and electrodermal signals of a patient subject to at least one anesthetic agent and at least one analgesic agent during an operative medical procedure. The system can also include a processor, operably coupled to the one or more sensors. The processor is configured to receive the EEG and electrodermal signals and, using the EEG or electrodermal signals, generate a report indicating the nociceptive state of the patient in real-time during the operative medical procedure while the patient is subject to the at least one anesthetic and a pain management plan based on desired post-operative outcomes.

### CROSS REFERENCE

This application is based on, claims priority to, and incorporates herein by reference in its entirety for all purposes, U.S. Provisional Patent Application Ser. No. 63/320,535, filed Mar. 16, 2022.

### TECHNICAL FIELD

The disclosure relates generally to the field of medicine and, more particularly, to the field of medical treatment planning and/or patient monitoring.

### BACKGROUND

Each year tens of thousands of patients die from opioid-related overdoses, making opioid-related overdoses one of the largest ongoing public health problems in the America. Surgery is a major risk factor for developing opioid dependence since the majority of surgical patients are prescribed opioids to manage their post-operative pain. Post-operative pain is strongly influenced by how well intraoperative nociception is controlled during surgery and general anesthesia. Therefore, effective control of nociception during general anesthesia and surgery is essential for mitigating post-operative pain and post-operative opioid requirements.

Surgical nociception is typically treated by administering opioid analgesics, but these drugs must be administered with care. Excessive opioid administration can induce respiratory depression and oversedation, increasing post-op length of stay, and can provoke central sensitization, which can increase downstream opioid requirements. On the other hand, ineffective control of surgical nociception can lead to increased postoperative pain, which would increase post-op opioid requirements. Currently, anesthesiologists rely on changes in heart rate (HR) and blood pressure (BP) to indicate nociception and titrate opioids and other drugs to minimize these changes. Unfortunately, HR and BP are influenced by numerous intraoperative factors, such as blood loss, anesthetic drugs, and anti-hypertensive medications, making them unreliable indicators of nociception. Improved methods to monitor surgical nociception during general anesthesia are therefore clearly needed. Fortunately, HR and BP are not the only physiological variables available to monitor and track nociception. Electrodermal activity (EDA), also known as skin conductance response (SCR), changes in response to nociceptive stimuli via autonomic mechanisms. Fluctuations in the electroencephalogram (EEG) can also track arousal and nociception. Recently, our lab has also identified a novel, robust, and specific EEG signature for opioid drugs that could be used to monitor opioid drug effects distinct from other anesthetic drugs.

Up to 10% of surgical patients develop opioid dependence after surgery. Acute postoperative pain that is poorly controlled can lead to chronic pain that may require long-term opioid use to control. Opioids are a first-line treatment of acute post-operative pain but are highly addictive. Anesthesiologists and ICU physicians and nurses do not have any tools to help them monitor their patients' nociception during surgery and/or during ICU care, nor the efficacy of the opioid pain medications they administer to treat nociception, leading to oversedation and sub-optimal post-operative pain outcomes.

### SUMMARY

The disclosure addresses the aforementioned drawbacks by describing systems and methods for monitoring patient parameters during medical procedures, such as intraoperatively, and/or for patent treatment planning to reduce the potential for undesired risks associated with some procedures or post-procedure care, such as when administering opioid drugs. In one non-limiting example, systems and methods are provided for patient planning and/or monitoring to better understand nociception and developing plans for management of post-operative use of drugs or other treatments, such as the administration of opioids. In one non-limiting example, a system may be used to that is configured to acquire patient information and determine signature in the information that are highly correlated with opioid drug concentrations and that can be applied in titrating opioids, independent of sedative hypnotic drugs, during general anesthesia and/or sedation. Such signature(s) may also be used alongside other physiological features, to monitor nociception and analgesia during anesthesia and intensive care. These features may be combined into a monitoring index that can be used to improve post-operative pain and opioid requirements. A surgical nociception monitor may be provided to reduce post-operative opioid requirements. In one further, non-limiting example, an integrated measure of nociceptive control based on neurophysiologic (as one non-limiting example, using EEG) and autonomic markers (as one non-limiting example, using EDA) of arousal and nociception provide anesthesiologists with a monitor that empowers clinicians to create plans, both surgical and/or post-surgical, that minimize post-operative pain and post-operative opioid requirements.

In one aspect of the present disclosure, an intraoperative patient monitoring system is described, the system comprising: one or more sensors configured to measure electroencephalogram (EEG) and electrodermal signals of a patient subject to at least one anesthetic agent and at least one analgesic agent during an operative medical procedure; and a processor, operably coupled to the one or more sensors, configured to: receive the EEG and electrodermal signals; using the EEG or electrodermal signals, monitor a nociceptive state of the patient in real-time during the operative medical procedure; and generate a post-operative pain management plan using at least one of the nociceptive state of the patient during the operative medical procedure or the at least one analgesic agent administered to the patient for the operative medical procedure.

In another aspect of the present disclosure, an intraoperative patient monitoring system is described, the system comprising: one or more sensors configured to measure electroencephalogram (EEG) and electrodermal signals of a patient subject to at least one anesthetic agent and at least one analgesic agent during an operative medical procedure; and a processor, operably coupled to the one or more sensors, configured to: receive the EEG and electrodermal signals; and using the EEG or electrodermal signals, generate a report indicating the nociceptive state of the patient in real-time during the operative medical procedure while the patient is subject to the at least one anesthetic.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIG. 1 shows an illustration of a closed-loop monitoring and control system in accordance with the present disclosure.

FIG. 2A shows the predicted effect site concentration (ESC) of a representative subject.

FIG. 2B shows the exposure to fentanyl corresponded with changes in the spectrogram.

FIG. 2C shows the exposure to fentanyl corresponded with the power spectrum changes in a representative subject, with notable increases in theta (4-8 Hz) and Slow/Delta (0-4 Hz) bands. The increases in Theta Power correspond to increase in fentanyl concentration.

FIG. 2D shows a mixed effects model that was constructed across subjects with 95% CI to further describe the association between changes in theta power and fentanyl concertation.

FIG. 3A shows a theta oscillation (4-8 Hz) that appears to be unique to fentanyl emerges during loss of consciousness. The theta band signal appears responsive to noxious stimuli, as it noticeably decreases during intubation.

FIG. 3B spectrogram a distinct change during loss of consciousness at 25 minutes and during intubation at 28 minutes.

FIG. 4A shows fentanyl concentrations estimated with a Pk/Pd model.

FIG. 4B shows that the theta band power correlates closely with reaction time across the protocol.

FIG. 4C is the evaluation prediction error through K-fold cross validation comparing theta power model the Pk/Pd model, and the combined model.

FIG. 5A is a traditional spectral analysis and state space methods shows EEG signatures on the slow, theta and alpha bands during sevoflurane and fentanyl general anesthesia.

FIG. 5B is an AIC analysis using the 2- and 3-oscillator state space models showing that the 3-oscillator model is consistently better than the 2-oscillator model, indicating that the fentanyl theta component can be detected and extracted against a background of sevoflurane-induced slow and alpha oscillations.

FIG. 6A shows an EEG spectrogram during surgery under general anesthesia, annotated to indicate time points when propofol is administered and when a lidocaine infusion is stopped.

FIG. 6B shows that the EDA SCR decreases appreciably after a bolus of propofol, consistent with the presence of an anti-nociceptive agent.

FIG. 6C shows that after surgery, a lidocaine infusion meant to provide analgesia is stopped; the patient remains unconscious, but the EDA SCR increases significantly, suggesting increased nociception.

FIG. 6D shows the evaluation of specific epochs for potential nociceptive stimuli via event records obtained from a patient's anesthesia record. Epochs with nociceptive stimuli showed increased SCR compared to baseline. This pattern was consistent during maintenance of general anesthesia, despite the reduced SCR level likely due to the presence of anesthetic drugs.

FIG. 7A are EEG spectrograms showing instances where patients have a consistent alpha band oscillations related to unconsciousness (left) vs disruptions in the alpha oscillations related to nociception/arousal (right).

FIG. 7B shows that the alpha-band oscillation of a patient's EEG recording is extractable using the previously described state space model, the components of which can be used to further explore disruptions during various stimuli, including the amplitude or envelope as shown, or the frequency or phase of the oscillation.

FIG. 8A shows that the alpha band power appears to decrease as the SCR increases during a lumbar puncture.

FIG. 8B is a spectrogram showing the alpha power fluctuations aligned with SCR response.

FIG. 8C shows a cross-correlation that was evaluated showing a similar association between alpha power and SCR across multiple nociceptive events and subjects (mean=0.5135, SD=0.0558).

FIG. 9A summary of prospective data collection for model, algorithm, and index development.

FIG. 10 shows a schematic comparing typical approaches for developing operating room monitors to the approach according to aspects of the present disclosure for developing an algorithm to optimize and calibrate intraoperative monitoring variable with respect to post-operative outcomes.

FIG. 11A shows the average fentanyl concentration during surgery in three representative patients.

FIG. 11B shows median theta EEG power during surgery in the same three representative patients.

FIG. 12A shows the total opioids required in the first post-operative 24 hours for the same three representative patients.

FIG. 12B shows the maximum pain experienced in the first post-operative 24 hours by the same three representative patients.

FIG. 13 shows the median skin conductance during the surgical period for the same three representative patients.

FIG. 14A shows the fluctuation in EEG alpha oscillation amplitude during the surgical period for the same three representative patients.

FIG. 14B shows the fluctuation in EEG alpha oscillation instantaneous frequency during the surgical period for the same three representative patients.

### DETAILED DESCRIPTION

Described below are systems and methods for monitoring nociception and analgesia using a combination of physiological measurements including electroencephalogram (EEG) and electrodermal activity (EDA). In a non-limiting example, separate sensors are used to acquired EEG and EDA data. In one embodiment, one or more EEG sensors are applied to a patient's scalp. Alternatively, the one or more EEG sensors are applied to a patient's forehead. In a non-limiting example, one or more EDA sensors are be applied on the surface of a patient's skin, such as the palms of the hand. In an embodiment the one or more EDA sensors are placed on a patient's forehead. In another embodiment, the EEG and EDA measurements may be obtained using a single sensor. In an embodiment, the single sensor for measuring EEG and EDA is placed on a patient's forehead.

The embodiments employ novel methods for extracting information from these signals related to a patient's autonomic responses to nociception, their cerebral responses to nociception, and to the patient's pharmacologic response to opioid analgesic drugs.

Commercial EEG-based anesthesia monitors have been on the marketplace for several decades. However, existing devices have focused on monitoring a patient's level of consciousness. Monitoring nociception (i.e., experiencing and physiologically responding to noxious stimuli), however, is something that existing technologies are unable to do. Some benefits of the aspects of the current invention include 1) use and processing of a novel EEG signature of opioid drugs, 2) use and processing of a novel sensor for electrodermal activity, 3) use and processing of a novel signature for cerebral responses to nociception, 4) integration of all of these features within a quantitative system to predict post-operative outcomes of interest including post-operative pain and opioid consumption, and 5) calibration of this integrated monitoring feature to minimize post-operative pain and opioid consumption.

We have performed three studies that support these concepts: 1) a prospective laboratory investigation in n=25 human patients designed to discover an opioid EEG signature; 2) a retrospective analysis of clinical EEG data screening thousands of surgeries under general anesthesia; 3) a retrospective cohort study including tens of thousands of cases examining the relationship between patient baseline variables, intraoperative opioid administration, and post-operative pain and opioid outcomes.

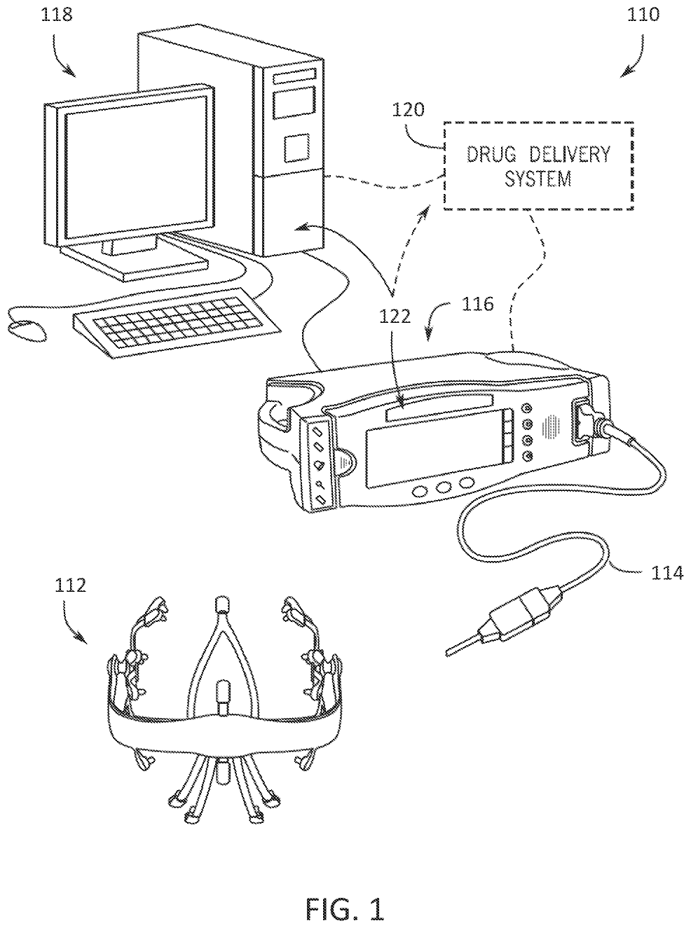

Referring to FIG. 1, an exemplary system 110 in accordance with the present invention is illustrated. The system 110 includes a patient monitoring device 112, such as a physiological monitoring device, which may include an electroencephalography (EEG) sensor array. However, it is contemplated that the patient monitoring device 112 may also include mechanisms for monitoring electrodermal activity (EDA), for example, to measure arousal to external stimuli or other monitoring system such as cardiovascular monitors, including electrocardiographic and blood pressure monitors. In a non-limiting example, EEG electrodes and EDA electrodes are integrated into the same sensor of the patient monitoring device 112. Alternatively, the EEG sensor array and EDA sensor array may be separate. In a preferred embodiment, the patient monitoring device 112 is placed on the surface of a patient's forehead. In an alternative embodiment, the EEG sensor array may be placed on the scalp or forehead and the EDA sensor array may be placed on the palms of the patient.

The patient monitoring device 112 is connected via a cable 114 to communicate with a monitoring system 116. Also, the cable 114 and similar connections can be replaced by wireless connections between components. As illustrated, the monitoring system 116 may be further connected to a dedicated analysis system 118. Also, the monitoring system 116 and analysis system 118 may be integrated.

In a non-limiting example, the monitoring system 116 may be configured to receive raw signals acquired by the combined EEG sensor and EDA sensor array, assemble, and display the raw signals as EEG waveforms, EEG spectrograms, and/or EDA skin conductance response (SCR) data. Accordingly, the analysis system 118 may receive the EEG waveforms from the monitoring system 116 and, as will be described, analyze the EEG waveforms and signatures therein based on a selected anesthesia compound, determine a state of the patient based on the analyzed EEG waveforms and signatures, and generate a report, for example, as a printed report or, preferably, a real-time display of signature information and determined state. However, it is also contemplated that the functions of monitoring system 116 and analysis system 118 may be combined into a common system.

In a non-limiting example, the analysis system 118 may further generate a post-operative pain management plan based on signals received from the patient monitoring device. As described in further detail below, effective site concentration (ESC) signatures of specific analgesic agents (e.g., opioids) may provide information about a patient's nociception state. Additionally, changes in nociceptive state are detected by changes in EDA, due to the neurally mediated effects on sweat gland permeability, which are observed as changes in the resistance of the skin to a small electrical current or as differences in the electrical potential between different parts of the skin.

Alternatively, in a non-limiting example, an intraoperative plan may be generated by a system that is calibrated to target and optimize post-operative outcomes including but not limited to post-operative pain, opioid requirements, cognitive recovery time, and respiratory depression. The system can be configured to characterize the relationship between intraoperative opioid administration and post-operative pain and opioid requirements. For example, a relationship may be represented by an increased intraoperative opioid administration and decreased post-operative pain, opioid requirements, respiratory depression, and/or length of stay in hospital.

In a non-limiting example, Table 1 provides a non-exhaustive list of the post-operative pain management outcomes, which may include a maximal pain score during a Post Anesthesia Care Unit (PACU), cumulative opioid dose administered during the PACU, frequency of uncontrolled pain at 24 hours, new instances of chronic pain diagnosis between 3 months and 1 year, total opioid use at 24 hours and in-hospital, opioid prescriptions at 30, 90, and 180 postoperative days, frequency of new persistent opioid use at 90 and 180 days, maximal pain score in the first 24 hours and in-hospital, incidence of opioid related complications in PACU (Postoperative nausea and vomiting (PONV), sedation, and respiratory depression), length of stay (LOS) in PACU and in-hospital, 30-day readmission, and 30-day mortality.

Table 1 shows the expected effect of the addition of 100 mcg fentanyl or 500 mcg hydromorphone to the observed intraoperative exposure of each patient in our study population. These analyses indicated that a 100-mcg increase in intraoperative fentanyl would correspond to a 0.26-point mean reduction in maximum pain score in the PACU, and a 500-mcg increase in intraoperative hydromorphone would result in a 0.12-point mean reduction in maximum pain score in the PACU.

For post-operative opioid administration, a 100-mcg increase in intraoperative fentanyl corresponds to mean reductions of 0.44 Morphine Milligram Equivalents (MME) post-op opioid administration in the PACU (−16.0%), 3.2 MME at 24 hours (−29.3%), and 6.6 MME in hospital (−14.5%). Meanwhile, a 500-mcg increase in intraoperative hydromorphone would correspond to mean reductions of 0.26 MME of opioid administration in the PACU (−9.3%), 1.9 MME at 24 hours (−17.7%), and 1 MME in hospital (−2.2%).

A 100-mcg increase in intraoperative fentanyl would correspond to an 8.2-hour reduction in hospital length of stay (−12.5%), whereas a 500-mcg increase in intraoperative hydromorphone would correspond to a 4.2-hour increase in hospital length of stay (+6.3%). For opioid prescriptions, a 100-mcg increase in intraoperative fentanyl would correspond to decreases of 20.7 instances per 1000 cases after 30 days (−8.3%), 22.6 instances per 1000 cases after 90 days (−8.6%), and 23.1 instances per 1000 cases after 180 days (−8.3%), alongside a decrease of 16.9 instances per 1000 cases of persistent opioid use (−10.2%). A 500-mcg increase in intraoperative hydromorphone would correspond to increases of 11.3 opioid prescriptions per 1000 cases after 30 days (+4.5%), 11.3 opioid prescriptions per 1000 cases after 90 days (+4.3%), and 11.2 opioid prescriptions per 1000 cases after 180 days (+4.0%), alongside an increase of 4.3 instances of persistent use per 1000 cases (+2.6%).

TABLE 1
Counterfactual predictions. For non-pain score
outcomes, expected change and 95% confidence
intervals are also reported as percentages.

| | with additional | with additional 500 mcg |
|---|---|---|
| Change in outcome | 100 mcg fentanyl | hydromorphone |
| (Primary) PACU | −0.26 | −0.12 |
| Max Pain Score | (−0.30, −0.21) | (−0.15, −0.09) |
| 24 h Postop | 0.12 | 0.01 |
| Max Pain | (0.07, 0.17) | (−0.02, 0.04) |
| In-hospital | 0.06 | 0.16 |
| Max Pain | (0.001, 0.11) | (0.14, 0.19) |
| (Primary) PACU | −0.44 | −0.26 |
| Total MME | (−0.45, −0.38) | (−0.31, −0.20) |
| | −16.0%, | −9.3%, |
| | (−18.1%, −13.8%) | (−11.2%, −7.3%) |
| 24 h Postop | −3.2 | −1.9 |
| Total MME | (−3.5, −2.9) | (−2.1, −1.8) |
| | −29.3%, | −17.7%, |
| | (−31.8%, −26.5%) | (−19.2%, −16.2%) |
| In-Hospital | −6.6 | −1.0 |
| Total MME | (−10.2, −4.2) | (−2.1, 0.3) |
| | −14.5%, | −2.2%, |
| | (−21.8%, −9.1%) | (−4.5%, 0.6%) |
| Recovery from | 0.29 | −0.09 |
| Sedation (min) | (−0.619, 1.48) | (−1.08, 0.84) |
| | 0.8%, | −0.3%, |
| | (−1.8%, 4.2%) | (−3.1%, 2.4%) |
| PACU Length of | −1.08 | −0.50 |
| Stay (min) | (−3.13, 0.027) | (−1.66, 0.25) |
| | −1.5%, | −0.7%, |
| | (−4.5%, 0.04%) | (−2.4%, 0.4%) |
| Hospital Length | −493.5 | 249.8 |
| of Stay (min) | (−823.1, −599.0) | (289.1, 427.3) |
| | −12.5%, | 6.3%, |
| | (−20.9%, −15.2%) | (7.3%, 10.8%) |
| Frequency of | −5.09 | 1.68 |
| Uncontrolled | (−6.90, −4.22) | (0.71, 3.13) |
| Pain (cases/1000) | −56.2%, | 18.5%, |
| | (−76.2%, −46.6%) | (7.9%, 34.6%) |
| 3-Month | −1.92 | 0.16 |
| Chronic Pain | (−2.79, −1.17) | (−0.39, 0.75) |
| (cases/1000) | −37.6%, | 3.2%, |
| | (−54.6%, −22.9%) | (−7.5%, 14.7%) |
| 12-Month | −0.96 | −3.150 |
| Chronic Pain | (−3.00, 1.17) | (−4.42, −1.97) |
| (cases/1000) | −5.2%, | −17.1%, |
| | (−16.3%, 6.3%) | (−24.0%, −10.7%) |
| 30-Day Opioid | −20.71 | 11.25 |
| Prescription | (−26.70, −14.62) | (7.33, 15.05) |
| (cases/1000) | −8.3%, | 4.5%, |
| | (−10.7%, −5.9%) | (2.9%, 6.0%) |
| 90-Day Opioid | −22.64 | 11.30 |
| Prescription | (−28.73, −16.18) | (7.27, 15.47) |
| (cases/1000) | −8.6%, | 4.3%, |
| | (−10.9%, −6.1%) | (2.7%, 5.8%) |
| 180-Day Opioid | −23.06 | 11.21 |
| Prescription | (−29.43, −16.63) | (7.17, 15.24) |
| (cases/1000) | −8.3%, | 4.0%, |
| | (−10.6%, −6.0%) | (2.6%, 5.5%) |
| Persistent Use | −16.94 | 4.30 |
| (cases/1000) | (−22.43, −11.53) | (0.99, 7.70) |
| | −10.2%, | 2.6%, |
| | (−13.5%, −6.9%) | (0.6%, 4.6%) |
| PONV in PACU | 2.50 | 8.21 |
| (cases/1000) | (−0.75, 6.11) | (5.21, 11.71) |
| | 8.0%, | 26.2%, |
| | (−2.4%, 19.5%) | (16.6%, 37.4%) |
| Respiratory | −3.58 | 1.15 |
| Depression in | (−5.27, −1.88) | (−0.80, 3.41) |
| PACU (cases/1000) | −28.1%, | 9.0%, |
| | (−41.3%, −14.7%) | (−6.2%, 26.8%) |
| 30-Day Postop | 0.24 | −0.91 |
| Mortality | (−0.51, 1.29) | (−1.49, −0.49) |
| (cases/1000) | 7.8%, | −29.3%, |
| | (−16.4%, 41.3%) | (−48.0%, −15.9%) |
| 30-Day Postop | −10.16 | 7.53 |
| Readmission | (−13.11, −6.98) | (5.67, 9.43) |
| (cases/1000) | −15.9%, | 11.8%, |
| | (−20.6%, −11.0%) | (8.9%, 14.8%) |

PACU: Post-Anesthesia Care Unit;
MME: Morphine Milligram Equivalents;
PONV: Post-Operative Nausea and Vomiting.

In a non-limiting example, the system may also be configured to select or identify at least one of the post-operative pain and opioid requirement variables with the highest correlation. The system may be further calibrated by restricting the post-operative pain and opioid requirement outcomes to acceptable ranges. Further, the system may be refined by a subspace and range of modifiable physiological variables that lead to acceptable post-operative outcomes.

As will be detailed, the system 110 may also include a drug delivery system 120. The drug delivery system 120 may be coupled to the analysis system 118 and monitoring system 116, such that the system 110 forms a closed-loop monitoring and control system. As will be described, such a closed-loop monitoring and control system in accordance with the present invention is capable of a wide range of operation but includes user interfaces 122 to allow a user to configure the closed-loop monitoring and control system, receive feedback from the closed-loop monitoring and control system, and, if needed reconfigure and/or override the closed-loop monitoring and control system.

In a non-limiting example, the drug delivery system 120 may include a plurality of specific subsystems to administer any one of anesthetic agents, analgesic agents (opioid and non-opioid), vasopressors, antihypertensive drugs, opioid antagonists, analgesics and anesthetic adjuncts, or combination thereof. It may also account for CYP3A4 inducers and inhibitors that may alter the dose response characteristics of any of the above-mentioned therapeutic drugs.

In a non-limiting example, anesthetic agents may include Nitrous Oxide, Propofol, Desflurane, Isoflurane, and Sevoflurane.

In a non-limiting example, opioid analgesic agents may include Fentanyl, Hydromorphone, Morphine, Methadone, Oxycodone, Meperidine, Remifentanil, Codeine, Hydrocodone, Oxymorphone, Sufentanil, Alfentanil, Nalbuphine, Buprenorphine, Butorphanol, Levorphanol, Pentazocine, Tramadol, Tapentadol, Dihydrocodeine, Opium, and Paregoric.

In a non-limiting example, non-opioid analgesic agents may include Aspirin, Celecoxib, Diclofenac, Diflunisal, Etodolac, Fenoprofen, Flurbiprofen, Ibuprofen, Indomethacin, Ketorolac, Ketoprofen, Magnesium salicylate, Meclofenamate, Mefenamic acid, Meloxicam, Nabumetone, Naproxen, Oxaprozin, Piroxicam, Salsalate, Sulindac, Tolmetin, Acetaminophen, Gabapentin, Pregabalin, Carbamazepine, Oxacarbamazepine, Valproic acid, Topiramate, Dexamethasone, Prednisone, Amitriptyline, Nortriptyline, Doxepin, Clomipramine, Duloxetine, Venlafaxine, Milnacipran, Desvenlafaxine, Lamotrigine, Cyclobenzaprine, Methocarbamol, Baclofen, Tizanidine, Clonidine, Propranolol, Verapamil, Almotriptan, Eletriptan, Frovatriptan, Naratriptan, Rizatriptan, Sumatriptan, Zolmitriptan, Ketamine, Lidocaine, Pamidronate, Zoledronic acid, Denosumab, Capsaicin, and Diclofenac.

In a non-limiting example, vasopressors may include, Dopamine (100 mcg/kg/min), Ephedrine, Epinephrine (1 mcg/kg/min), Norepinephrine (1 mcg/kg/min), Phenylephrine (10 mcg/kg/min), and Vasopressin (0.4 mcg/kg/min).

In a non-limiting example, CYP3A4 inducers may include Apalutamide, Carbamazepine, Enzalutamide, Fosphenytoin, Lumacaftor, Lumacaftor-Ivacaftor, Mitotane, Phenobarbital, Phenytoin, Primidone, Rifampin, Rifampicin, Bexarotene, Bosentan, Cenobamate, Dabrafenib, Dexamethasone, Efavirenz, Elagolix, Eslicarbazepine, Etravirine, Lorlatinib, Modafinil, Nafcillin, Pexidartinib, Rifabutin, Rifapentine, St. John's Wort, Nevirapine, and Griseofulvin.

In a non-limiting example, CYP3A4 inhibitors may include Atazanavir, Ceritinib, Clarithromycin, Cobicistat, Darunavir, Idelalisib, Indinavir, Itraconazole, Ketoconazole, Lonafarnib, Lopinavir, Mifepristone, Nefazodone, Nelfinavir, Ombitasvir-paritaprevir-ritonavir, Ombitasvir-paritaprevir-ritonavir-dasabuvir, Posaconazole, Ritonavir, Saquinavir, Tucatinib, Voriconazole, Amiodarone, Aprepitant, Berotralstat, Cimetidine, Conivaptan, Crizotinib, Cyclosporine, Diltiazem, Duvelisib, Dronedarone, Erythromycin, Fedratinib, Fluconazole, Fosamprenavir, Fosaprepitant, Fosnetupitant-Palonosetron, Imatinib, Isavuconazole, Isavuconazonium Sulfate, Lefamulin, Letermovir, Netupitant, Nilotinib, Ribociclib, Verapamil, and Metronidazole.

In a non-limiting example, Antihypertensive Drugs May Include Esmolol, Metoprolol, Propranolol, Labetalol, Nicardipine, Clevidipine, Hydralazine, Nitroglycerin, Glyceryl Trinitrate, Nitroprusside, Fenoldopam, Verapamil, and Diltiazem.

In a non-limiting example, opioid antagonists may include Naxolone, Naltrexone, Methylnaltrexone, and Alyimopam.

In a non-limiting example, analgesics and anesthetic adjuncts may include Diclofenac, Ibuprofen, Indomethacin, Ketorolac, Meloxicam, Acetaminophen, Lidocaine, Ketamine, Dexmedetomidine, Esmolol, Magnesium (sulfate), and Dexamethasone.

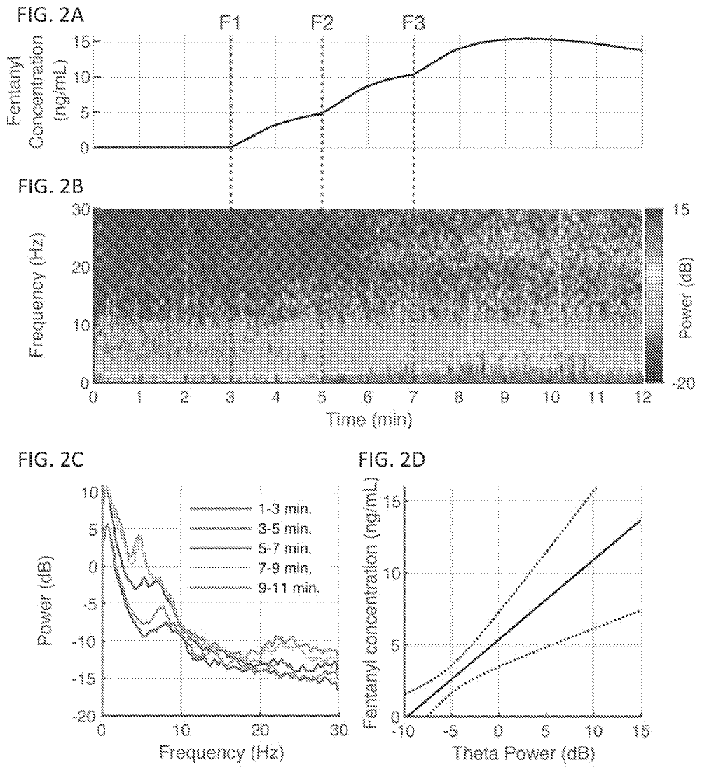

In a non-limiting example, FIGS. 2A-2D show the administration and EEG signature for fentanyl. Subjects (n=25) were administered up to 3 boluses in 2-minute intervals after a baseline period, and concentration level were computed through pharmacokinetic/pharmacodynamic (Pk/Pd) modeling. The changes in EEG signal with ESC of fentanyl are shown in FIG. 2B. The fentanyl effect site concentration (ESC) is estimated using pharmacokinetic/pharmacodynamic (PK/PD) modeling, illustrated in FIG. 2A for a representative subject. The EEG may be analyzed using multitaper spectral analysis. A spectrogram from a representative subject in FIG. 2B illustrates how EEG power in the theta (4 to 8 Hz) and slow/delta (0 to 4 Hz) bands increases as the fentanyl concentration increases. These same oscillations may also be appreciated in the spectrum, illustrated at different time points in FIG. 2C. In FIG. 2D, a strong association across all n=25 subjects [slope: 0.55 (CI: 0.25 to 0.80), marginal R2=0.151, conditional R2=0.744] between the EEG and fentanyl concentration is shown in a linear mixed-effects model representing fentanyl concentration as a function of theta power. As illustrated in FIG. 2D, for a fentanyl effect site concentration that ranges between 0 and approximately 15 ng/mL, the theta power may range between approximately −10 to 15 dB.

Further details of the systems and methods are described in the following examples.

### EXAMPLE

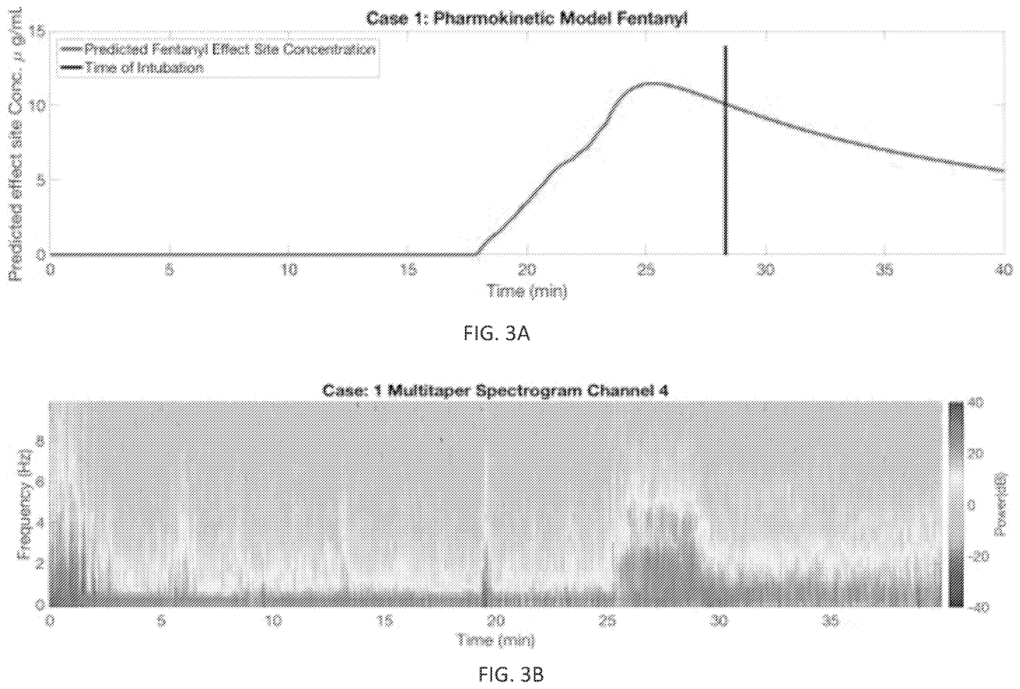

1.a. A novel opioid-specific EEG signature that tracks patient state and that is detectable during general anesthesia (polypharmacy). Retrospective EEG data from n=6 patients receiving fentanyl for induction of general anesthesia during cardiac surgery and discovered a novel fentanyl-induced EEG signature. FIGS. 3A-3B show spectral features unique to fentanyl sedation and unconsciousness. More particularly, FIGS. 3A-3B show the EEG spectrogram from a representative subject, showing an increase in the slow-(0.1-1 Hz), delta-(1-4 Hz) and theta-band power (4-8 Hz) coinciding with an anesthetized state. The theta band signal appears to be a distinct oscillation unique to fentanyl.

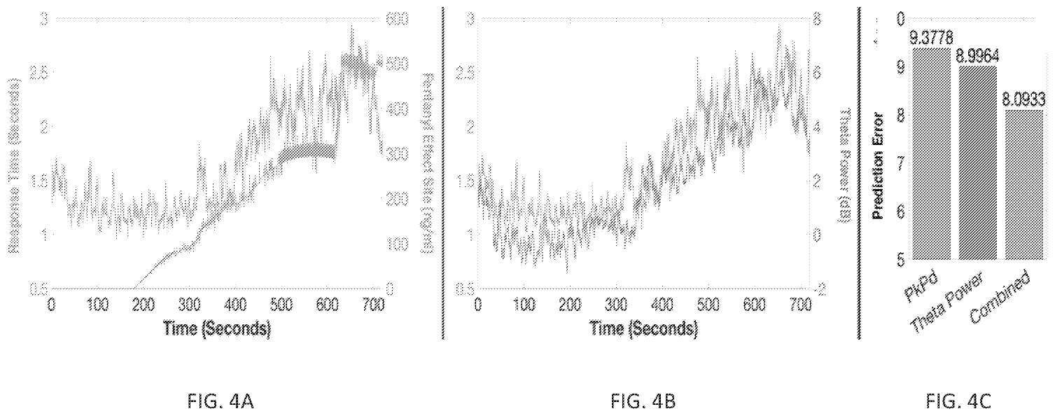

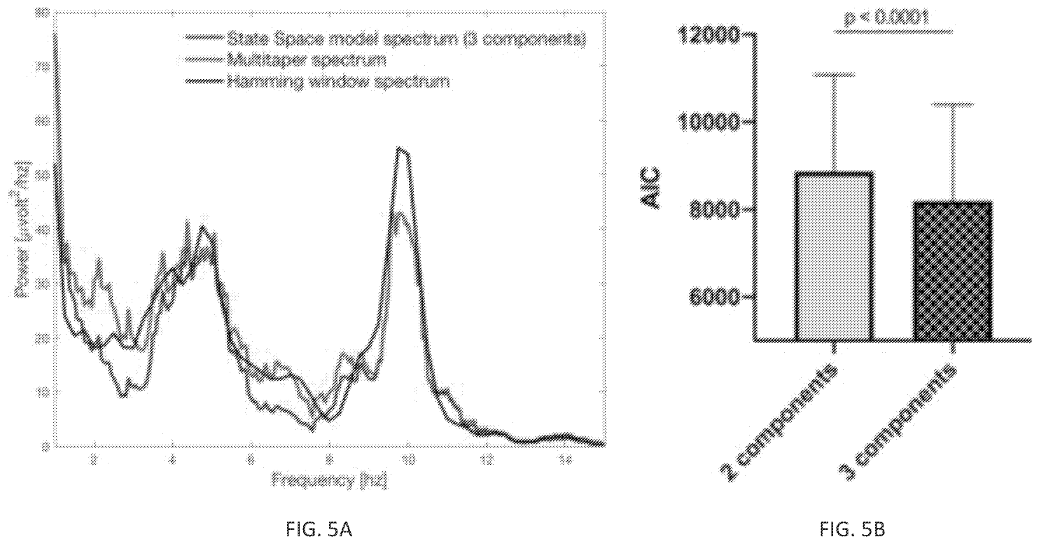

Prospectively recorded EEG data in n=26 who received fentanyl prior to induction of general anesthesia alongside a structured auditory behavioral response task was performed. After a 5-minute baseline period, patients were administered two or three boluses of 2 mcg/kg ideal body weight fentanyl separated by 2 minutes (as in FIG. 2A). FIGS. 4A-4C are a visualization of models using fentanyl Pk/Pd concentrations and theta power models to predict behavioral response times. In FIG. 4A, patients' response times to a repeated auditory stimulus were used to gauge the patients' level of awareness. FIG. 4B shows how the EEG theta power signature tracks patients' behavioral response times. How the EEG theta signature might perform as a predictor of behavioral response in comparison to and combination predicted fentanyl Pk/Pd effect site concentration was investigated (FIG. 4C). Linear regression models were fit using either fentanyl (predicted effect site) concentration, EEG theta concentration, or both fentanyl and EEG theta combined, characterizing model mean-squared prediction error (MSE) using k-fold cross validation in which one patient was omitted from each cross-validation run. The EEG theta and combined models had lower MSE than the fentanyl effect site concentration model by 4% and 14%, respectively. The combined model, using all data, had an MSE at P<<0.001 using a generalized likelihood ratio test. These data indicate that EEG theta power, alone or in combination with drug information, is a strong predictor of opioid-induced patient states, better than the predicted Pk/Pd effect site concentration alone. EEG theta power, again either by itself or in combination with drug information, may provide information on an individual patient's real-time, personalized drug response to opioid analgesics.

In a non-limiting example, it was investigated whether this fentanyl-induced EEG theta signature could be extracted during general anesthesia (polypharmacy), when other drugs such as propofol or sevoflurane are being administered to maintain unconsciousness. In particular, propofol and sevoflurane induce large slow and alpha oscillations that could obscure the nearby fentanyl theta oscillation. A model comparison analysis was performed to determine whether two (slow, alpha) or three (slow, theta, alpha) oscillations were present in the data. A class of “state space oscillator” models was used with either two or three oscillators and an AIC to compare models. In on non-limiting example, a state space oscillator model provided by Matsuda and Komaki (2017) and Beck, He, Gutierrez, and Purdon (2022) can be utilized. FIG. 5A shows the EEG spectrum in a representative subject during sevoflurane and fentanyl general anesthesia analyzed using traditional spectral analysis and state space methods. All methods clearly show peaks in the slow, theta, and alpha bands. FIG. 5B shows the AIC analysis using the 2- and 3-oscillator state space models (FIG. 5B) over n=30 subjects receiving sevoflurane and fentanyl. This indicates that the fentanyl theta component can be detected and extracted against a background of sevoflurane-induced slow and alpha oscillations. In other retrospective analyses (not shown) a similar theta oscillation when remifentanil was used in combination with propofol was found. These data support the ability to use the opioid EEG signature as a marker of nociceptive control and opioid drug effect during general anesthesia or other scenarios when multiple drugs are administered in addition to opioids.

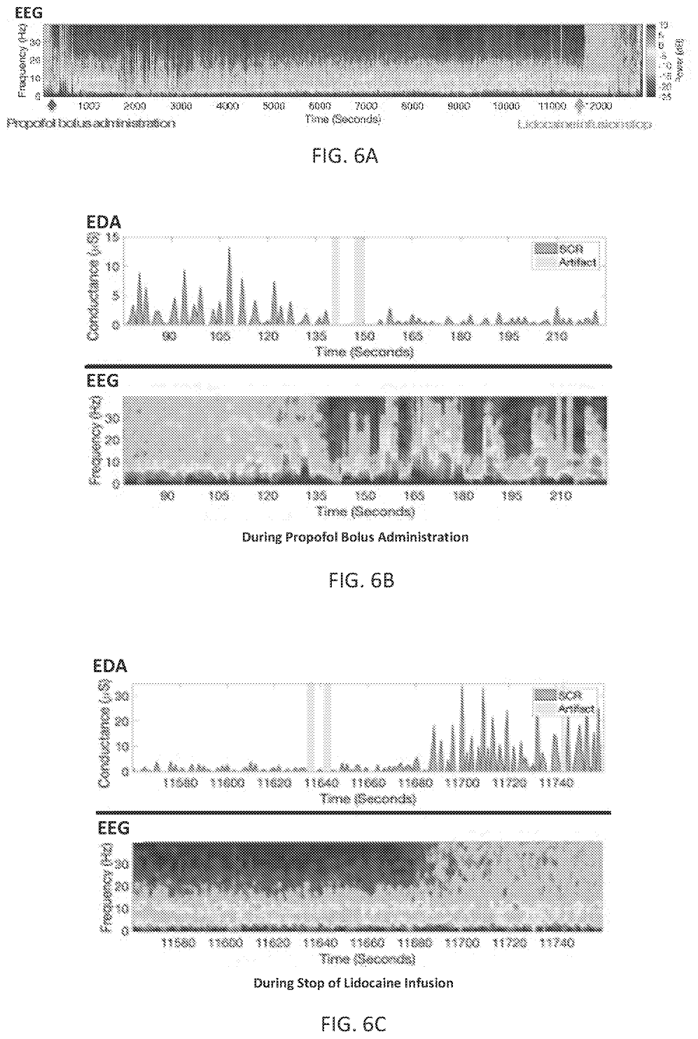

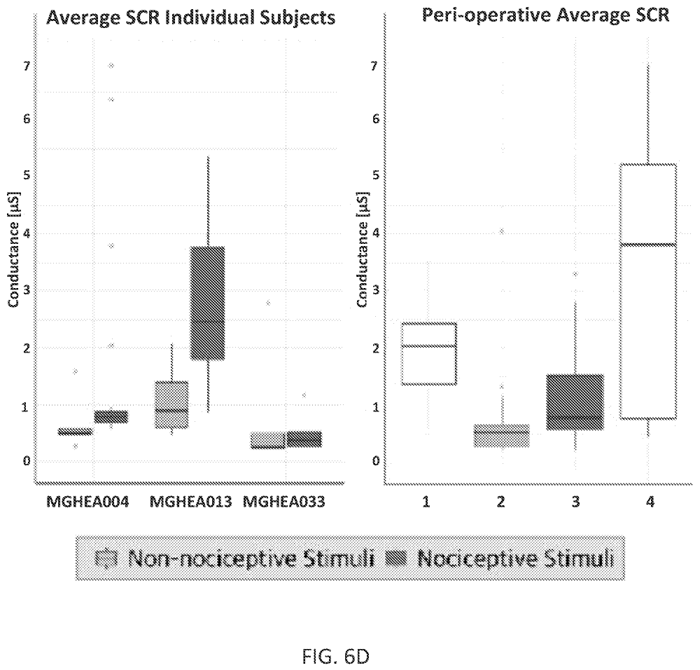

1.b. Electrodermal activity recorded from forehead sensors, simultaneously with EEG, tracks surgical nociception. EDA tracks autonomic changes provoked by nociceptive or affective stimuli. Typically, EDA are recorded from palmar surfaces that have high densities of sweat glands. The forehead, however, also has a high density of sweat gland comparable to the palms and EDA could be measured there at the same time as EEG. This may be accomplished by any number of methods that are well-known in the field, including for example administering a known electrical current across the EEG electrode, measuring the resulting voltage change, and inferring the skin conductance. FIGS. 6A-6D are an observation of skin conductance response (SCR) at nociception events during surgery. That is, EDA information was measured from forehead sensors simultaneously with EEG during general anesthesia, as shown in FIGS. 6A-6C. FIGS. 6B-6C show representative forehead EDA data from a single subject receiving general anesthesia during surgery. In FIG. 6B the EDA skin conductance response (SCR) decreases appreciably after induction of general anesthesia with propofol and remains below approximately 5 micro-Siemens thereafter. In FIG. 6C, a lidocaine infusion meant to provide analgesia is stopped; the patient remains unconscious, but the EDA SCR increases appreciably, above the approximately 5 micro-Siemens maintained during surgery and general anesthesia, rising to levels as high as approximately 35 micro-Siemens, suggesting increased nociception. In FIG. 6D, in three representative subjects, EDA levels were compared before, during, and after surgery. During surgery, suspected nociceptive events (e.g., intubation, first incision, active surgery) showed appreciably larger SCR than non-nociceptive periods (e.g., prior to active surgery start). EDA decreases during general anesthesia but increases after emergence. These preliminary data show that it is possible to simultaneously record EDA and EEG from the forehead, and confirm that EDA changes track surgical nociception with a combined sensor.

In a non-limiting example, the simultaneous monitoring of EEG and EDA intra-operatively may inform intraoperative fentanyl dose titration. The reduction in SCR after induction of general anesthesia with propofol in FIG. 6B and the SCR increase after lidocaine infusion in FIG. 6C may suggest an upper range of fentanyl titration. For example, a conductance below a predetermined value, for instance, below 5 micro-Siemens, may indicate an optimal intra-operative fentanyl dose to minimize intra-operative nociception and post-operative pain. Alternatively, the optimal fentanyl does may correspond to a range of conductance values.

1.c. Fluctuations in alpha oscillation amplitude track arousal and correlate with EDA; Novel methods to precisely extract alpha fluctuations. Changes in anesthesia-induced frontal alpha band (8-12 Hz) power and amplitude are known to change with changes in arousal: Alpha power increases with increasing anesthetic doses from sedation through unconsciousness and decreases during arousal and/or noxious stimulation.

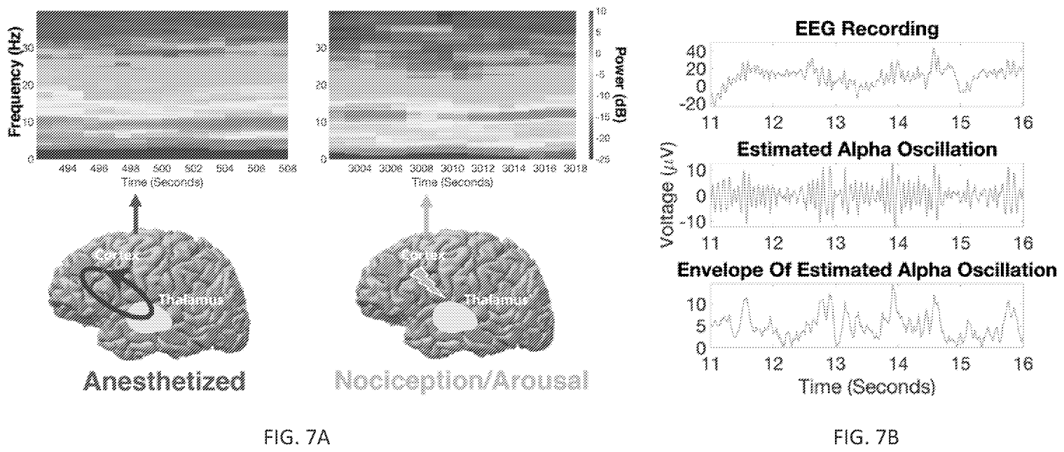

FIGS. 7A-7B show the extraction of alpha oscillations and alpha amplitude in surgical cases during general anesthesia. Changes in alpha power can therefore be used to track nociception-related arousal. FIG. 7A shows a typical EEG spectrogram during general anesthesia where the alpha power is fluctuating, presumably due to underlying changes in arousal or nociception. Some state space signal processing methods have been developed that can extract instantaneous fluctuations in alpha amplitude (FIG. 7B), as described in Beck, He, Gutierrez, and Purdon (2022), as well as phase and instantaneous frequency information. Alternatively, other standard methods could also be employed such as applying a bandpass filter for alpha frequencies, performing a Hilbert transform, and employing the bandpass (real) and Hilbert transformed (imaginary) signals to estimate the alpha amplitude, phase, or instantaneous frequency. Fluctuations in the alpha power or alpha frequency or alpha amplitude may indicate nociception-related arousal and may be used to titrate opioid or other non-opioid analgesic drugs, for instance by selecting a dose that minimizes fluctuations in alpha power, frequency, or amplitude.

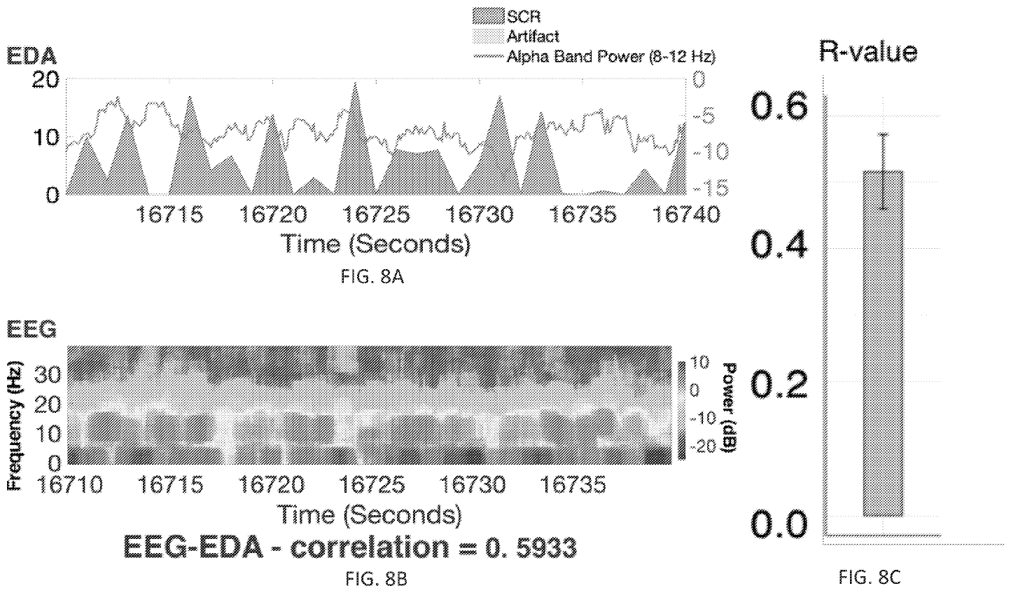

FIGS. 8A-8C are a comparison of the skin conductance response (SCR) against the alpha band power. that are highly correlated with simultaneously recorded EDA (FIGS. 8A-8B), in both individual subjects and on average across the three patients studied in FIG. 6D. The correlation between alpha amplitude and EDA helps corroborate previous literature linking alpha fluctuations to nociception (FIG. 8C).

In a non-limiting example, in order to generate an intraoperative nociception management plan to optimize post-operative outcomes using the disclosed system and methods, the monitoring index or variables are constructed and calibrated using models that characterize the relationship between intraoperative variables, anesthetic drug information, patient baseline and demographic variables, and post-operative outcomes including but not limited to post-operative pain, opioid requirements, cognitive recovery time, and respiratory depression. Such models may be constructed using any number of approaches including, but not limited to, machine learning models, deep learning networks, or regression models. These models may take into account additional confounding variables or covariates that may introduce bias into the prediction of the post-operative outcomes, including patient baseline, demographic, medical history, anesthetic record and other clinical variables, which may be obtained from an electronic health record system. In a non-limiting example, data acquired including all of the above measurements and variables and/or collected from patients in a systematic fashion is used to construct and calibrate such models.

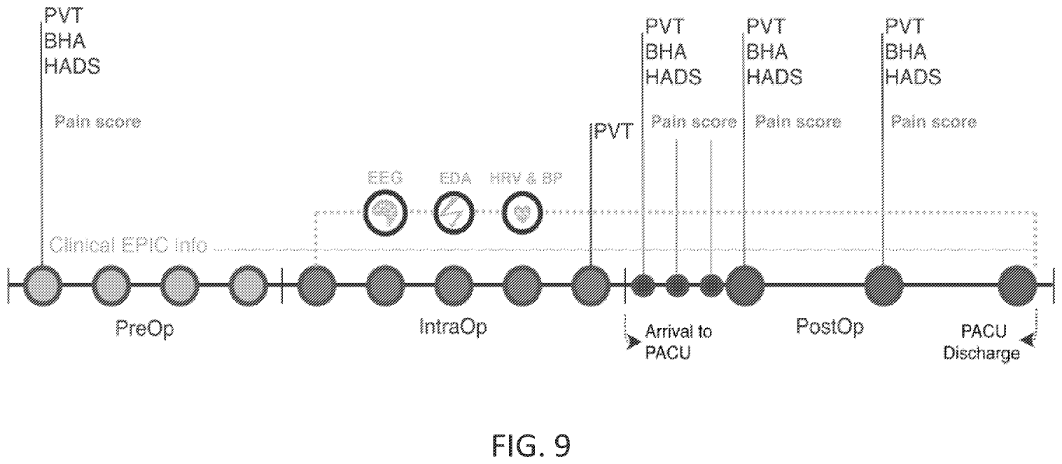

FIG. 9 shows a schematic describing the variables to be measured and their timeline within a clinical observational study designed to collect data for model, algorithm, and index development. Pre-operative measurements include Pain Numeric Rating, Psychomotor Vigilance, TabCAT Brain Health Assessment (BHA), and hospital anxiety and depression scale (HADS). Intra-operative measurements include EEG and EDA, as well as heart rate variability (HRV), blood pressure (BP) waveform data and records of all medications and clinical events. Post-operatively in the post-operative care unit (PACU), measurements include EEG and EDA recording continue, alongside Pain scores every 15 mins for the first hour and every hour after that. PVT, BHA and HADS are measured hourly until PACU discharge. Opioid related side-effects, Modified Aldrete score (respiratory depression), PACU and inpatient pain medications and opioids administered, and their 90 and 180-day opioid consumption status are from medical records. Patient baseline, demographic, medical history, anesthetic record and other clinical variables may be obtained from an electronic health record system (e.g. EPIC).

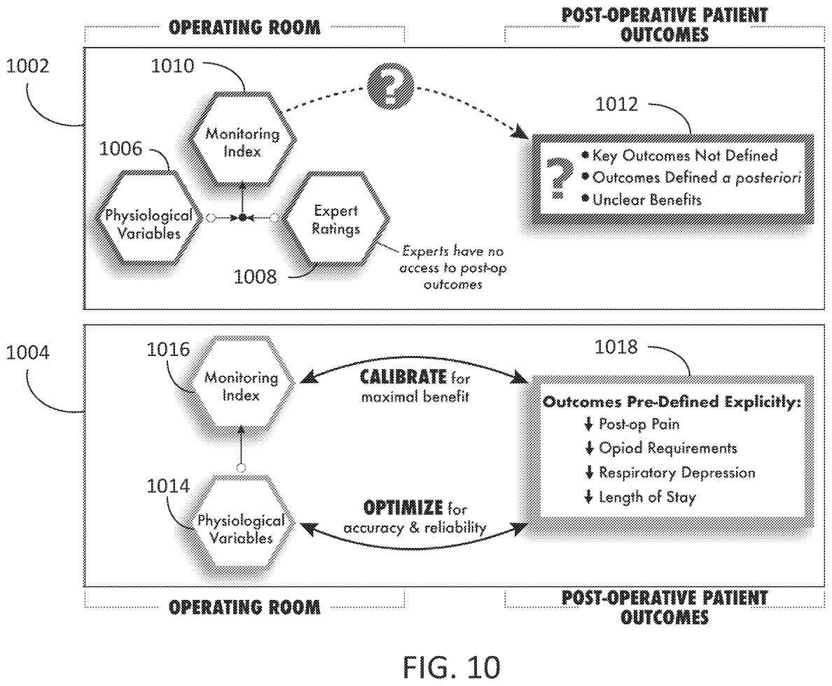

Referring to FIG. 10, whereas some approaches 1002 to developing operating room monitors have focused on physiological variables 1006 and expert ratings 1008 in the operating room setting, they do not explicitly account for the relationship of these intraoperative variables to post-operative outcomes 1012 in the construction or calibration of the monitoring system, algorithm, or monitoring index 1010. Consequently, operating room monitors developed in this typical approach 1002 may not be able to achieve desired post-operative outcomes 1012, since the monitoring index 1010 values or ranges of such values may bear no or little relationship to the desired post-operative outcomes 1012. Alternatively, the approach described according to aspects of the present disclosure 1004, employs data obtained, for example, as described in FIG. 9, to operate the monitoring system, algorithms, and monitoring index 1016 derived from physiological variables 1014 and patient records to achieve the desired post-operative outcomes 1018. As described herein, a post-operative plan can be delivered that is based on the operative process to decrease post-operative pain, opioid requirements, respiratory depression, and length of post-operative hospital stay compared to post-operative plans that do not consider the opioids or other drugs administered during the operative procedure.

In one non-limiting example, the systems and the methods of the present disclosure were considered relative to three female patients, Patient 1, Patient 2, and Patient 3, aged 48, 51, and 52, respectively, each receiving surgery under general anesthesia with varying levels of intraoperative analgesia and different post-operative outcomes. For each patient, the average fentanyl concentration is computed, median theta EEG power, skin conductance, and fluctuations in alpha EEG amplitude and instantaneous alpha EEG frequency during the surgical period. Average fentanyl concentration across the surgical period was computed by extracting dosage information from the electronic medical record system and employing the Pk/Pd model described by McClain and Hug (Clin Pharmacol Ther 1980) to calculate the effect site concentration. Theta power across the surgical duration were computed with the multitaper spectral estimation method in four-second windows across the surgical duration. Tapers centered at 6 Hz covering the theta band of 4 to 8 Hz were used to estimate theta power in each window, and the median of these values across the surgical duration was reported. Electrode conductance was estimated by demodulating a 7.5 nA, 78 Hz test current used to measure electrode impedance. The data were filtered with a 6-Hz bandwidth filter and then the Hilbert transform was applied to estimate the amplitude of the 78 Hz signal, which in turn was used to compute the impedance and the conductance. The median of these values across the surgical duration were reported. Alpha amplitude and instantaneous frequency were computed by applying the Hilbert transform to the EEG data bandpass filtered to 8-12 Hz with a 2 Hz filter transition window. For each patient, post-operative outcomes were also examined, namely, the total opioids used in the first post-operative 24-hours and the maximum pain score in the first post-operative 24-hours, obtained from the electronic health record.

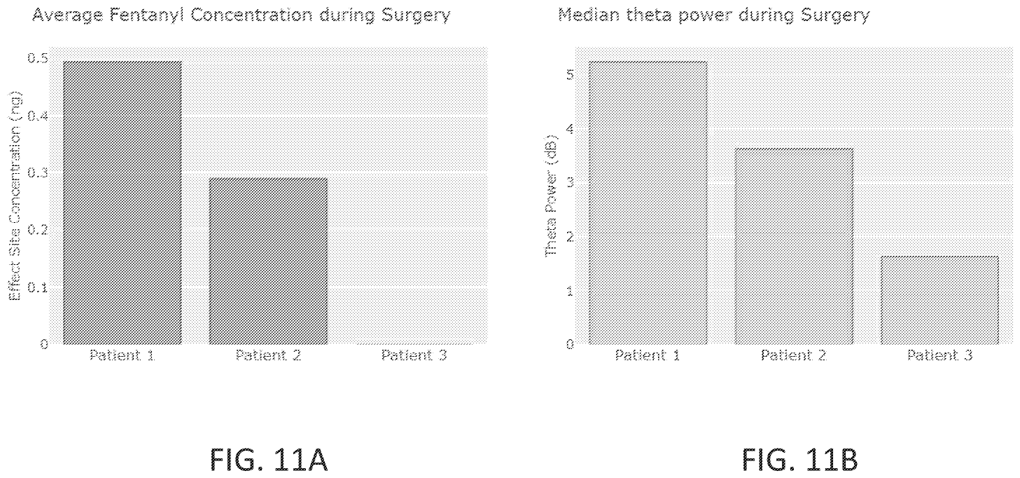

FIG. 11 shows for patients 1, 2, and 3, respectively, the average fentanyl concentration as well as the median theta power. Patient 1 was administered the highest fentanyl concentration, Patient 2 received a lower concentration of fentanyl, while Patient 3 received no fentanyl. The median theta power for each patient shows a corresponding trend for each patient, as one would expect from FIG. 2D, in which Patient 1 has the highest theta power, Patient 2 has lower theta power, and Patient 3 has the lowest theta power.

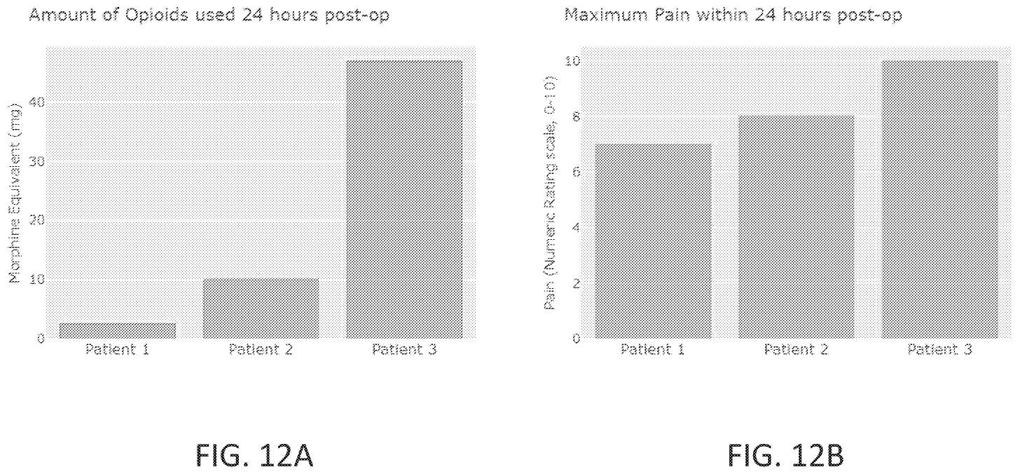

FIG. 12 shows post-operative outcomes for these patients. Consistent with the modeling and counterfactual analysis presented above, Patient 1, who had the highest fentanyl concentration and corresponding highest theta power, required the lowest amount of post-operative opioids for pain control in the first post-operative 24 hours of the three patients, and also exhibited the lowest post-operative 24-hour maximum pain score of the three patients. Patient 3, who had the lowest fentanyl concentration and lowest corresponding theta power, required the highest amount of post-operative opioids for pain control in the first post-operative 24 hours of the three patients, and also exhibited the highest post-operative 24-hour maximum pain score of the three patients. Patient 2, who had a fentanyl concentration and corresponding theta power between those of Patients 1 and 3, required an amount of post-operative opioids for pain control in the first post-operative 24 hours between the amounts required by Patients 1 and 3, and exhibited a post-operative 24-hour maximum pain score between those exhibited by Patients 1 and 3. As illustrated in FIG. 4, theta power, alone or in combination with drug concentrations estimated using Pk/Pd models, provide an accurate assessment of individual personalized patient response to opioids. Considering the modeling and counterfactual analysis presented earlier, the theta power, alone or in combination with estimated drug concentrations, could be used to predict post-operative outcomes or guide analgesia to attain desired post-operative outcomes.

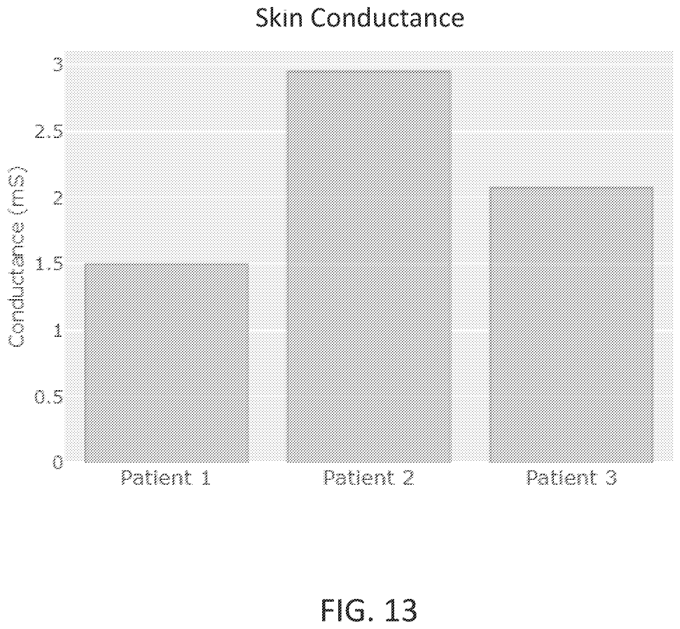

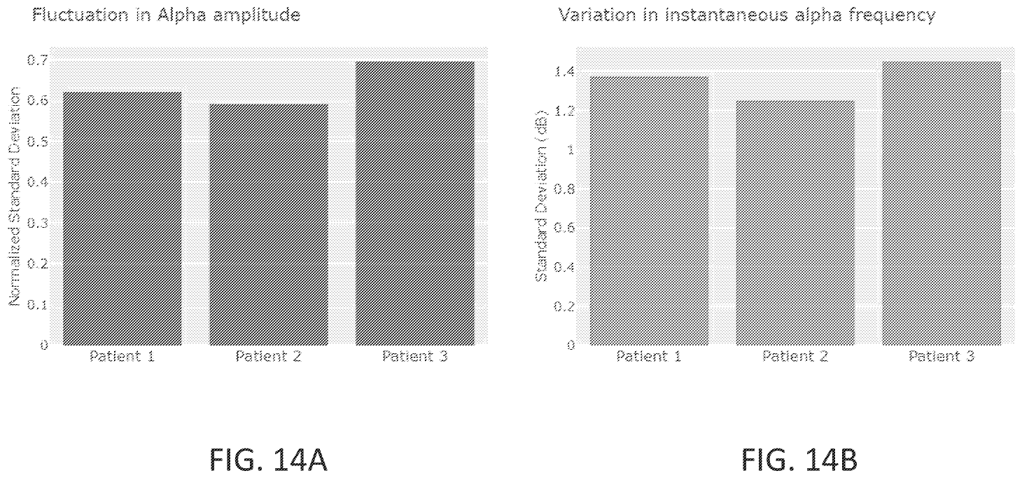

Each patient's skin conductance is shown in FIG. 13. Consistent with earlier descriptions, maintaining the skin conductance below some threshold may help optimize post-operative pain and opioid requirements. Each patient's fluctuations in alpha amplitude and alpha instantaneous frequency are shown in FIG. 14. Consistent with earlier descriptions, maintaining these fluctuations in alpha amplitude and alpha instantaneous frequency below some threshold may help optimize post-operative pain and opioid requirements.

Given the plurality of relevant measurements including opioid-induced EEG theta oscillations, skin conductance, alpha-band EEG amplitude and instantaneous frequency fluctuations, alongside a plurality of complex patient baseline variables, medical history, and multiple drugs being administered, among other variables, a system and method according to aspects of the present disclosure to model relationships among these variables and summarize information to guide intraoperative analgesia or post-operative pain management would be advantageous and highly desirable. In contrast, given the complexity of this high dimensional information that must often be interpreted in real-time, clinicians would be unlikely to consistently attain optimal outcomes in the absence of such a system and a method to process this plurality of information.

It will be appreciated by those skilled in the art that while the disclosed subject matter in connection with particular embodiments and examples, the invention is not necessarily so limited, and that numerous other embodiments, examples, uses, modifications and departures from the embodiments, examples and uses are intended to be encompassed by the claims attached hereto. Various features and advantages of the invention are set forth in the following claims.

1. An intraoperative patient monitoring system, the system comprising:
one or more sensors configured to measure electroencephalogram (EEG) and electrodermal signals of a patient subject to at least one anesthetic agent and at least one analgesic agent during an operative medical procedure; and
a processor, operably coupled to the one or more sensors, configured to:
receive the EEG and electrodermal signals;
using the EEG or electrodermal signals, monitor a nociceptive state of the patient in real-time during the operative medical procedure; and
generate a post-operative pain management plan using at least one of the nociceptive state of the patient during the operative medical procedure or the at least one analgesic agent administered to the patient for the operative medical procedure.

2. The system of claim 1, wherein the processor is further configured to identify from the EEG signal a signature highly correlated with an effective site concentration (ESC) in the patient of the at least one analgesic agent.

3. The system of claim 1, wherein the at least one analgesic agent includes an opioid.

4. The system of claim 3, wherein the opioid includes fentanyl.

5. The system of claim 1, wherein the post-operative pain management plan includes a plurality of post-operative outcomes.

6. The system of claim 5, wherein the plurality of outcomes includes at least one of a post-operative pain, opioid requirements, cognitive recovery time, or respiratory depression.

7. The system of claim 1 wherein the processor is further configured to achieve a desired post-operative outcome including at least one of reduced post-operative pain, opioid requirements, cognitive recovery time, and respiratory depression based on the nociceptive state of the patient in real-time during the operative medical procedure.

8. An intraoperative patient monitoring system, the system comprising:
one or more sensors configured to measure electroencephalogram (EEG) and electrodermal signals of a patient subject to at least one anesthetic agent and at least one analgesic agent during an operative medical procedure; and
a processor, operably coupled to the one or more sensors, configured to:
receive the EEG and electrodermal signals; and
using the EEG or electrodermal signals, generate a report indicating the nociceptive state of the patient in real-time during the operative medical procedure while the patient is subject to the at least one anesthetic.

9. The system of claim 8, wherein the processor is further configured to identify from the EEG signal a signature highly correlated with an effective site concentration (ESC) in the patient of the at least one analgesic agent.

10. The system of claim 9, wherein the at least one analgesic includes an opioid.

11. The system of claim 10, wherein the opioid includes fentanyl.

12. The system of claim 9, wherein the report indicating the nociceptive state of the patient in real-time during the operative medical procedure further includes instruction to titrate at least one of the dose of the at least one anesthetic agent or at least one analgesic agent administered to the patient.
